(19) United States
(12) Patent Application Publication
(10) Pub. No.: US 2021/0187286 A1
(43) Pub. Date: Jun. 24, 2021

(54) Devices and Methods For Low-Latency Analyte Quantification Enabled By Sensing In The Dermis

(71) Applicant: Biolinq, Inc., San Diego, CA (US)

(72) Inventors: Joshua Windmiller, Del Mar, CA (US); Jared Rylan Tangney, Encinitas, CA (US); Thomas Arnold Peyser, Menlo Park, CA (US)

(73) Assignee: Biolinq, Inc., San Diego, CA (US)

(21) Appl. No.: 17/073,331

(22) Filed: Oct. 17, 2020

Related U.S. Application Data
(63) Continuation-in-part of application No. 16/824,700, filed on Mar. 20, 2020.
Application No. 16/824,700 is a continuation-in-part of application No. 16/666,259, filed on Oct. 28, 2019.
Application No. 16/666,259 is a continuation of application No. 16/152,372, filed on Oct. 4, 2018, now Pat. No. 10,492,708.
Application No. 16/152,372 is a continuation of application No. 15/590,105, filed on May 9, 2017, now Pat. No. 10,092,207.
(60) Provisional application No. 62/823,628, filed on Mar. 25, 2019.
(60) Provisional application No. 62/336,724, filed on May 15, 2016.
(60) Provisional application No. 62/927,049, filed on Oct. 28, 2019.

(51) Int. Cl. A61N 1/30; A61N 1/05; A61B 5/1468; A61B 5/05; A61B 5/145; A61M 5/172

(52) U.S. Cl. CPC A61N 1/30; A61N 1/05; A61B 5/1468; Y02E 60/50; A61B 5/14546; A61M 5/1723; A61B 2562/125; A61B 5/05

(57) ABSTRACT

Devices and methods for low-latency analyte quantification enabled by the implementation of a microneedle-based analyte-selective sensor operating in the dermis or viable epidermis are disclosed herein. The sensing element of the device is contained within the microneedle-based analyte-selective sensor and configured to penetrate the stratum corneum of the skin and become positioned in the viable epidermis or dermis of the wearer such that the sensing element is located a spatial distance no greater than 500 micrometers from the plexus of the dermis of the wearer.

### CROSS REFERENCES TO RELATED APPLICATIONS

The Present Application claims priority to U.S. Provisional Patent Application No. 62/927,049, filed on Oct. 28, 2019, is a continuation-in-part application of a U.S. patent application Ser. No. 16/824,700, filed on Mar. 20, 2020, which claims priority to U.S. Provisional Patent Application No. 62/823,628, filed on Mar. 25, 2019, now expired, and is a continuation-in-part application of U.S. patent application No. 16/666,259, filed on Oct. 28, 2019, which is a continuation application of U.S. patent Ser. No. 16/152,372, filed on Oct. 4, 2018, now U.S. Pat. No. 10,492,708 issued on Dec. 3, 2019, which is a continuation application of U.S. patent Ser. No. 15/590,105, filed on May 9, 2017, now U.S. Pat. No. 10,092,207, issued on Oct. 9, 2018, which claims priority to U.S. Provisional Patent Application No. 62/336,724, filed on May 15, 2016, now expired, each of which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The present invention generally relates to sensors.

### Description of the Related Art

The presentation of circulating biomarkers in a timely fashion remains a key aim in modern medical devices and chronic disease management, in particular. The most pertinent example of the need for low-latency biomarker or analyte quantification resides within the diabetes management domain and is addressed with continuous glucose monitoring systems (CGM or CGMS), which are widely used by individuals with insulin-dependent diabetes mellitus in order to inform dosing decisions involving the delivery of insulin. Automated insulin delivery systems are represented by closed-loop actuation mechanisms combining both the glucose sensing and therapeutic delivery mechanisms in order to modulate dosage of insulin without requiring user intervention. This paradigm embodies considerable potential to improve patient health outcomes; however, the success of such systems is largely predicated on their ability to quickly detect and react to changing interstitial glucose concentrations.¹ This is of particular importance when recovering from bouts of hypoglycemia as rapid transients can affect decisions to continue or abstain from therapy. Even in scenarios whereby the system operates as an open loop (i.e. requiring user intervention) or where the user self-administers insulin by means of an injection, the timely presentation of interstitial glucose concentrations is key to better managing the disease and maximizing time within a healthy glycemic window.

The prior art discusses various devices and methods.

Hayter et al., U.S. Pat. No. 9,770,211 for an Analyte sensor with time lag compensation, discloses methods and devices and systems for determining an analyte value.

Yang et al, U.S. Pat. No. 8,870,763 for a Method and/or system for multicompartment analyte monitoring, discloses monitoring and/or controlling levels of an analyte in bodily fluid. In particular, estimation of a concentration of the analyte in a first physiological compartment based upon observations of a concentration of the analyte in a second physiological compartment may account for a latency in transporting the analyte between the first and second physiological compartments.

McGarraugh et al., U.S. Pat. No. 8,216,138 for a Correlation of alternative site blood and interstitial fluid glucose concentrations to venous glucose concentration, discloses a method for calibrating an analyte-measurement device that is used to evaluate a concentration of analyte in bodily fluid at or from a measurement site in a body. The method involves measuring a concentration, or calibration concentration, of an analyte in blood from an “off-finger” calibration site, and calibrating the analyte-measurement device based on that calibration concentration. The invention also relates to a device, system, or kit for measuring a concentration of an analyte in a body, which employs a calibration device for adjusting analyte concentration measured in bodily fluid based on an analyte concentration measured in blood from an “off-finger” calibration site.

Brauker et al., U.S. Pat. No. 9,420,965 for a Signal processing for continuous analyte sensor, discloses Systems and methods for dynamically and intelligently estimating analyte data from a continuous analyte sensor, including receiving a data stream, selecting one of a plurality of algorithms, and employing the selected algorithm to estimate analyte values. Additional data processing includes evaluating the selected estimative algorithms, analyzing a variation of the estimated analyte values based on statistical, clinical, or physiological parameters, comparing the estimated analyte values with corresponding measure analyte values, and providing output to a user. Estimation can be used to compensate for time lag, match sensor data with corresponding reference data, warn of upcoming clinical risk, replace erroneous sensor data signals, and provide more timely analyte information encourage proactive behavior and preempt clinical risk.

Hayter et al., U.S. Pat. No. 9,332,934 for a Analyte sensor with lag compensation, discloses methods, devices and systems including calibrating analyte data associated with a monitored analyte level received from an analyte sensor based on a reference measurement, determining a lag time constant associated with the calibrated analyte data, and performing lag correction of the calibrated analyte data based on the determined time lag constant.

Feldman et al., U.S. Patent Publication Number 20150243851, for a Shallow implantable analyte sensor with rapid physiological response, discloses methods and devices to detect analyte in body fluid are provided. Embodiments include analyte sensors designed so that at least a portion of the sensor is positionable beneath a skin surface in the dermal layer.

Current subcutaneously-implanted analyte-selective sensors are configured to execute the analyte sensing operation in the subcutaneous layer beneath the dermis, known as the subcutaneous adipose tissue. This layer is the predominant location of triglyceride reserve (i.e. fat) storage in the human body. As a physiological adaptation arising from metabolically preferred sources of energy, which include extracellular glucose in the circulatory system and glycogen found within the body's muscles and liver, the subcutaneous adipose region not as well vascularized as other more metabolically-active physiological compartments. Accordingly, the subcutaneous compartment does not exhibit the same level of perfusion with circulating metabolites as other locations (bodily organs) requiring more immediate energy requirements. The dermis of the skin is one example of a bodily organ that requires rapid access to metabolically-favorable sources of energy (i.e. glucose and glycogen) in order to impart thermoregulation and barrier protection properties. Owing to the execution of the sensing operation in the poorly-vascularized subcutaneous layer beneath the dermis, current needle-, cannula-, and wire-based analyte-selective sensors are encumbered with a significant amount of physiological lag time, otherwise referred to as delay or latency.

Prior art solutions have largely been concerned with the implementation of various algorithmic methods² to reduce the preponderance of physiological lag associated with execution of the analyte sensing operation in the subcutaneous adipose tissue. These solutions aim at predicting the glycemic response in the immediate future (5-30 minutes), albeit are quite ineffective when an inflection point occurs in circulating glucose levels.

### BRIEF SUMMARY OF THE INVENTION

The technology described herein relates to the construction and operation of analyte-selective sensors and methods for configuration of the same for physiological sensing of analytes with reduced latency.

The current invention teaches of devices and methods for low-latency analyte quantification enabled by the implementation of a microneedle-based analyte-selective sensor operating in the dermis or viable epidermis. By operating the microneedle-based analyte-selective sensor in the dermis or viable epidermis, access to an analyte (i.e. glucose) or plurality of analytes circulating therein is facilitated. Furthermore, owing to the high level of vascularization and perfusion of the dermal strata and the reduced diffusion distance between the network of capillaries disposed within this region and the location of the analyte-selective sensor, rapid quantification of the analyte or plurality of analytes is achieved.

One aspect of the present invention is a method for the measurement of at least one physiological analyte with reduced latency. The method includes positioning an analyte-selective sensor on the skin of a wearer. The method also includes deploying the analyte-selective sensor such that a sensing element contained within the analyte-selective sensor penetrates the stratum corneum of the skin and becomes positioned in the viable epidermis or dermis of the wearer. The method also includes applying a voltage or current at the sensing element. The method also includes measuring one or more physiological analytes with the sensing element such that the measurement operation occurs no greater than 500 micrometers from the plexus of the dermis of the wearer.

Another aspect of the present invention is a device for the measurement of at least one physiological analyte with reduced latency. The device comprises an analyte-selective sensor and a sensing element. The analyte-selective sensor is configured to be positioned on the skin of a wearer. The sensing element is contained within the analyte-selective sensor and configured to penetrate the stratum corneum of the skin and become positioned in the viable epidermis or dermis of the wearer such that the sensing element is located a spatial distance no greater than 500 micrometers from the plexus of the dermis of the wearer.

The analyte-selective sensor is preferably an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

The analyte-selective sensor preferably comprises a microneedle or microneedle array that preferably has a vertical extent between 200 and 2000 μm. The sensing element is preferably confined to a region between 1 and 1500 μm from a distal end of the microneedle. The sensing element is preferably an electrode, a transducer, a detector, an anode or a cathode.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 illustrates prior art needle-/cannula-based analyte-selective sensor (left) configured for the quantification of glucose in the subcutis and a microneedle array-based analyte-selective sensor (right) configured for the quantification of glucose in the dermis in size reference to a dime coin.

FIG. 2 illustrates a microneedle array-based analyte-selective sensor with a sensing element (electrode) located in a distal region of the analyte-selective sensor and is intended to execute the measurement operation in the viable epidermis or dermis.

FIG. 3 is a pictorial representation of a conventional wire-/needle-/cannula-based analyte-selective sensor configured to operate within the subcutaneous tissue (left) and an analyte-selective sensor configured to operate within the dermis (right). It should be noted that the sensing element contained within the analyte-selective sensor (right) is located in the papillary dermis.

FIG. 3A is a pictorial representation of a conventional wire-/needle-/cannula-based analyte-selective sensor configured to operate within the subcutaneous tissue (right) and an analyte-selective sensor configured to operate within the dermis (left). It should be noted that the sensing element contained within the analyte-selective sensor (left) is located in the papillary dermis.

FIG. 4 is a pictorial representation of a cross-section of the skin delineating the anatomical location of the papillary plexus and structures contained therein.

FIG. 5 is a pictorial representation of a cross-section of the skin delineating the anatomical location of the superficial and dermal plexus and structures contained therein.

FIG. 6 is a process flow diagram illustrating a method of the invention.

FIG. 7 is a pictorial representation of a cross-section of the skin delineating the anatomical location of the superficial and dermal plexus and structures contained therein.

FIG. 8 is a cross-sectional view of a microneedle.

FIG. 8A is a cross-sectional view of a microneedle.

FIG. 9 is an isolated top perspective view of a microneedle array.

FIG. 9A is a top plan view of the microneedle array of FIG. 9.

FIG. 9B is side elevation view of a microneedle array of FIG. 9.

FIG. 9C is a bottom plan view of the microneedle array of FIG. 9.

FIG. 9D is front elevation view of a microneedle array of FIG. 9.

### DETAILED DESCRIPTION OF THE INVENTION

Current subcutaneously-implanted analyte-selective sensors have enjoyed much use in continuous physiological monitoring, driven primarily by the challenge of glucose quantification for diabetes applications. Configured to engage in the measurement of physiological analytes in the subcutaneous layer beneath the dermis, these analyte-selective sensors rely primarily on passive diffusion of the said analytes from the capillary bed found in the dermis. For sound physiological reason, the subcutaneous layer, which comprises adipose tissue, is the location in which the body stores a large portion of its fat reserves and hence does not require extensive perfusion, vascularization or a dense network of capillaries to sustain the cells therein. This is in contrast to the dermis, which is very well perfused and features a dense network of capillaries to sustain the cells therein, provide rapid immunological response, as well as mediate the human body's process of thermoregulation. The capillary wall is somewhat perm-selective, implying that analytes below a certain critical molecular mass (approx. <600 Da) can traverse through the capillary walls as a result of the interplay between hydrostatic pressure (directed from the capillary to the surrounding interstitial medium) and osmotic pressure (directed from the interstitial medium to the capillary). In this fashion, analytes of relatively low molecular mass are thus able to enter the interstitial fluid that bathes the cells in the dermis, otherwise known as the dermal extracellular matrix. Given the empirical relation for diffusion time:

$$t = \frac{{x}^{2}}{2 D}$$

where x refers to the distance traversed by a diffusing molecule and D represents the diffusion coefficient for said molecule in a host medium, one can easily ascertain that the diffusion time increases as the square of the distance between the source of a molecule (or analyte) and the detector (analyte-selective sensor). As can be inferred, the diffusion length of analytes traversing through the dermis and into the subcutaneous adipose tissue is relatively long, resulting in a delay between levels of an analyte in the circulatory system and when they ultimately diffuse to the subcutaneous sensor for detection. Another perspective of this phenomena may be gleaned from an inspection of the simplified one-dimensional time-dependent diffusion equation:

$$\frac{\partial C ( x , t )}{\partial t} = D {∇}^{2} C ( x , t )$$

where C refers to the concentration of a collection of diffusing molecules (the “analyte”), x refers to the distance traversed by said diffusing molecule ensemble, t is time, and D represents the diffusion coefficient for the said diffusing molecule in a host medium. Analytical solutions to this equation take the form:

$$C ( x , t ) = \frac{{C}_{0}}{\sqrt{4 π Dt}} exp ( - \frac{{x}^{2}}{4 Dt} )$$

where C₀ refers to the initial concentration of analyte at t=0 and x=0. As can be inferred from this relation, for a given time t, the shorter the distance x traveled by the diffusing analyte, the greater the equivalency between the concentration C(x,t) of the analyte at the current time to the initial concentration C₀ of said diffusing analyte. This demonstrates the merits of measuring an analyte in the vicinity of its source in order to make the closest possible determination of its actual concentration at the point of origin. Likewise, if the diffusion distance x is a very large figure, a substantial amount of time t must transpire before the measured quantity will resemble the original quantity at the point of origin at t=0.

As can be inferred, a subcutaneously-implanted analyte sensor succumbs to the forces of physiology—the increased diffusion distance between the source of the analyte (the dermal capillary bed) and the location of the sensing element in the subcutaneous adipose layer of tissue—thus resulting in a significant amount of measurement latency or lag time. Indeed, an analyte sensor can be designed with a very rapid intrinsic response to fluctuating analyte levels, albeit by positioning the said analyte sensor in the subcutaneous layer, the sensor remains latent for extended periods of time due to the intrinsic physiological diffusion of analytes through tissue. It should be mentioned that convection (i.e. mixing) may also contribute to the transfer of analyte from one physiological region to another. However, as this process notably difficult to control in vivo, its effect is frequently ignored and is oftentimes manifested as a transient artefact in the transduced analyte-selective sensor's signal.

The most pertinent example of the importance of the assessment of circulating analyte levels resides in the management of diabetes, where quantification of glucose in a continuous fashion has been shown to substantially improve outcomes via maximization of the time at healthy glucose levels, known as euglycemia. Furthermore, the timely quantification of circulating glucose levels in an individual with diabetes would make noteworthy inroads to addressing the challenge of meal detection or onset of physical activity, each of which can modulate circulating glucose levels to a significant degree and may require an intervention. In a number of cases, delivery of interventions in the acute phase would lessen the likelihood of the subject requiring a more involved treatment regimen or potentially experiencing serious complications. Another benefit of reduced latency in diabetes management is the identification of recovery from hypoglycemia as rapidly as possible. Indeed, the titration of carbohydrates is the most precarious during hypoglycemia and the timely identification of carbohydrate absorption in the acute phase could reduce the occurrence of overcompensated hypoglycemia, which oftentimes results in hyperglycemia that must be treated with pharmacologic agents, such as insulin.

Conventional subcutaneously-implanted needle-, cannula-, and wire-based analyte-selective sensors have been designed to engender maximal retention, signal strength, and compatibility with bulk manufacturing processes rather than the minimization of diffusional time lag. The current solution addresses the challenge of high latency engendered by these conventional analyte-selective sensors via physical placement of the analyte-selective sensor in the vicinity of the vascular layer of the dermis, known as the plexus, for optimal perfusion and minimal diffusion distance, thereby reducing sensor lag time. This ability is enabled by the implementation of microneedle-based analyte-selective sensors, which are configured to penetrate the stratum corneum when applied and conduct the analyte-selective sensing operation in the viable epidermis or dermis. In doing so, the analyte sensor is able to exploit the reduced amount of time that must transpire before an analyte originating from the circulatory system diffuses to and interacts with the said analyte sensor. Indeed, the microneedle-mediated sensing concept, which embeds analyte-selective sensors within the said microneedles/microneedle arrays, exploits the intrinsic advantages of the unique physiological region in which the analyte sensing regimen is executed. Furthermore and perhaps more profoundly, the analyte diffusion time increases as the square of the distance between the source of the said analyte (vascular layer of the dermis, which consists of a dense network of capillaries) and the position of the analyte-selective sensor, implying a second-order power-function temporal lag characteristic as the analyte-selective sensor is positioned further away from the source. As an example, using the empirical relation for diffusion time:

$$t = \frac{{x}^{2}}{2 D}$$

where the analyte of interest is glucose (D˜2.24×10⁻¹⁰ m²·s⁻¹), positioning an analyte-selective sensor at a distance of 1000 μm from the capillary layer (typical of current subcutaneous analyte-selective sensors) will result in a diffusion time of over 2200 seconds in the absence of any external perturbation. However, should the placement of the analyte-selective sensor be reduced to 100 μm from the capillary (typical of microneedle analyte-selective sensors), a diffusion time of only 22 seconds is required for transport of the analyte from the source (capillary) to the said analyte-selective layer. In reality, hydrostatic pressure tends to convect a diffusing analyte such that mass transfer occurs on smaller time scales. Nevertheless, there is a significant advantage of sensing an analyte in the local vicinity of its source. This advantage is uniquely provided by the micron-scale features that are the hallmark of microneedle-based analyte-selective sensors operating in the viable epidermis or dermis, but can be extended to any shallowly-implanted analyte-selective sensor operating in said physiological compartments.

Moreover, the current solution addresses the challenge of high latency engendered by conventional subcutaneously-implanted needle-, cannula-, and wire-based analyte-selective sensors via the implementation of microfabrication techniques, such as semiconductor processing or microelectromechanical systems (MEMS), in order to fabricate analyte-selective sensors with a sufficient degree of precision, thereby enabling precise placement of said analyte-selective sensors in the viable epidermis or dermis, hence reducing sensor lag time. Conventional subcutaneously-implanted needle-, cannula-, and wire-based analyte-selective sensors, on the other hand, are fabricated with bulk manufacturing methods and cannot achieve the level of precision required for insertion into the viable epidermis or dermis.

An analyte-selective sensor is a microneedle or microneedle array-based electrochemical, electrooptical, or fully electronic device configured to measure an endogenous or exogenous biochemical agent, metabolite, drug, pharmacologic, biological, or medicament in the dermal interstitium, indicative of a particular physiological or metabolic state in a physiological fluid of a user. Specifically, said microneedle array contains a plurality of microneedles, possessing vertical extent between 200 and 2000 μm, configured to selectively quantify the levels of at least one analyte located within the viable epidermis or dermis and in the vicinity of the papillary plexus, subpapillary plexus, or dermal plexus.

A sensing element is an electrode, transducer, detector, anode, or cathode where the analyte-sensing routine occurs. Said sensing element is configured to be deployed, penetrating the stratum corneum of the skin of a wearer and become positioned in the viable epidermis or dermis of said wearer such that said sensing element is located a spatial distance no greater than 500 micrometers from the plexus of the dermis.

In a method of the present invention, at a first step, a user applies sensor to the skin. The analyte-selective sensor is configured to adhere onto the skin of a wearer. At a second step, the sensing element contained within the sensor is deployed. The sensing element is configured to penetrate the stratum corneum of the skin and becomes positioned in the viable epidermis or dermis of said wearer. At a next step, measurement of an analyte or plurality of analytes in a selective fashion occurs. Once deployed, sensing element engages in the measurement of one analyte or a plurality of analytes such that said measurement operation occurs no greater than 500 micrometers from the plexus of the dermis of said wearer.

The inputs of the invention are a diffusing analyte or plurality of diffusing analytes originating from the plexus of the dermis. An analyte is a biological molecule or aggregation of biological molecules capable of traversing through the capillary wall into the dermal interstitial space. The outputs of the invention include a measurement of the concentration of a diffusing analyte or plurality of diffusing analytes. The outputs of the invention can also include an assessment of the presence of a diffusing analyte or plurality of diffusing analytes.

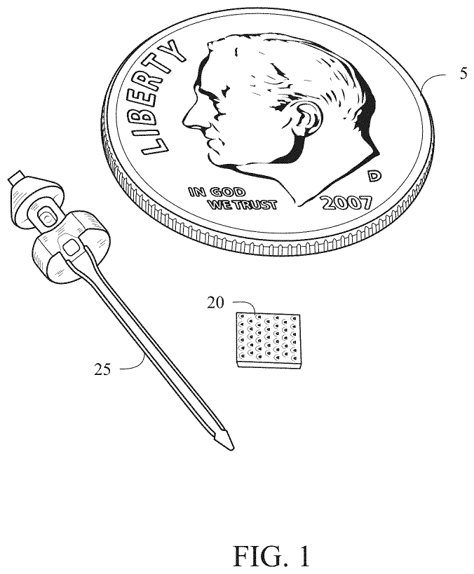

FIG. 1 illustrates prior art needle-/cannula-based analyte-selective sensor 25 configured for the quantification of glucose in the subcutis and a microneedle array-based analyte-selective sensor 20 configured for the quantification of glucose in the dermis in size reference to a dime coin 5.

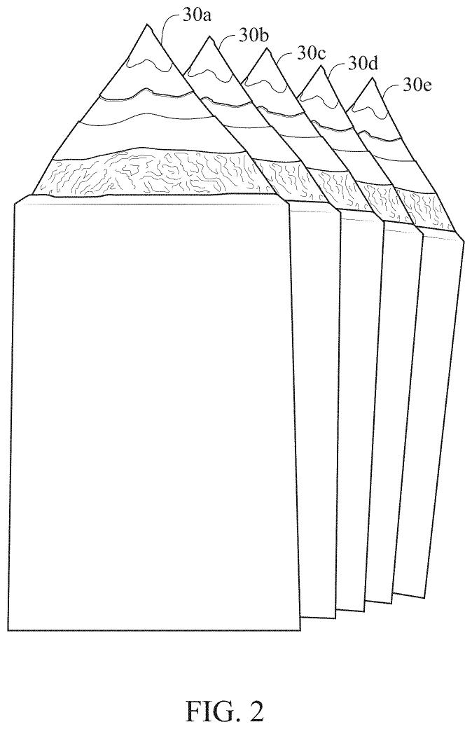

FIG. 2 illustrates microneedles 30a-30e of a microneedle array-based analyte-selective sensor.

As confirmed by scanning laser confocal microscopy, the average skin layer thickness requiring penetration includes the: Stratum corneum: 29±4 μm; Epidermis: 82±15 μm; Papillary dermis: 122±23 μm; Full thickness=233±42 μm. According to the preponderance of the scientific literature, microneedles employed for therapeutic interventions typically insert to a depth equivalent to ½-⅔ of their full height. A target height, therefore, is 350-466 μm. The microneedles 30 most preferably have a height of approximately 300-700 μm.

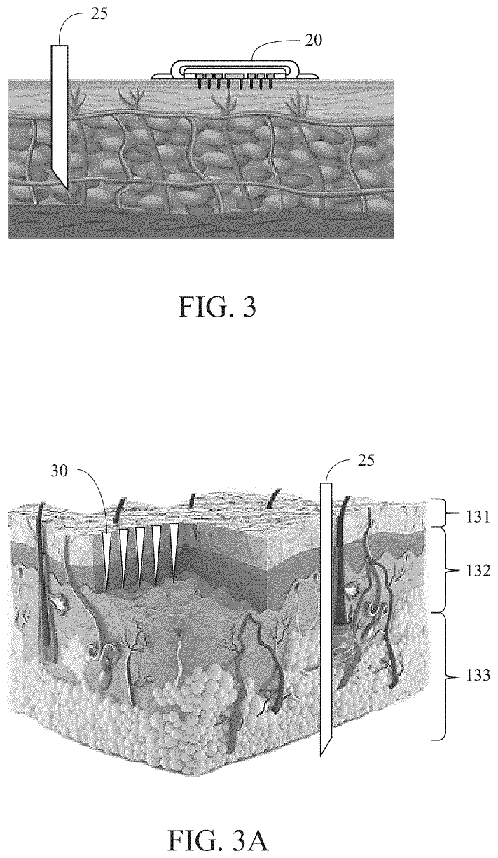

FIGS. 3 and 3A are pictorial representations of a conventional wire-/needle-/cannula-based analyte-selective sensor 25 configured to operate within the subcutaneous tissue 133 (10,000 microns or more) and an analyte-selective sensor 20 of the present invention configured to operate within the dermis 132, below the epidermis 131. The sensing element 31 (not shown) on the microneedle 30 contained within the analyte-selective sensor 20 is located in the papillary dermis.

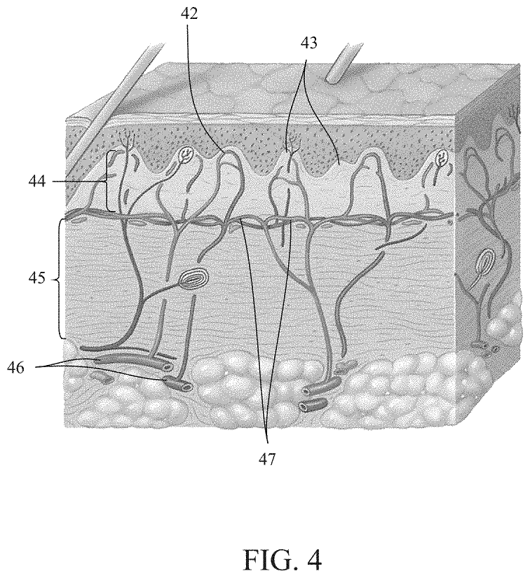

FIG. 4 is a pictorial representation of a cross-section of the skin delineating the anatomical location of the papillary plexus 47 and structures contained therein a capillary loop of papillary plexus 42, the dermal papillae 43, the papillary layer 44, the reticular layer 45, and the cutaneous plexus 46.

The microneedles 30 are designed to access papillary dermis and the interstitial fluid therein. The papillary dermis is adjacent to dermal plexus allowing for minimal diffusional latency from a capillary to a sensor. Also, the present design allows for reduced pain sensation owing to a lack of pain receptors in such a superficial anatomical region.

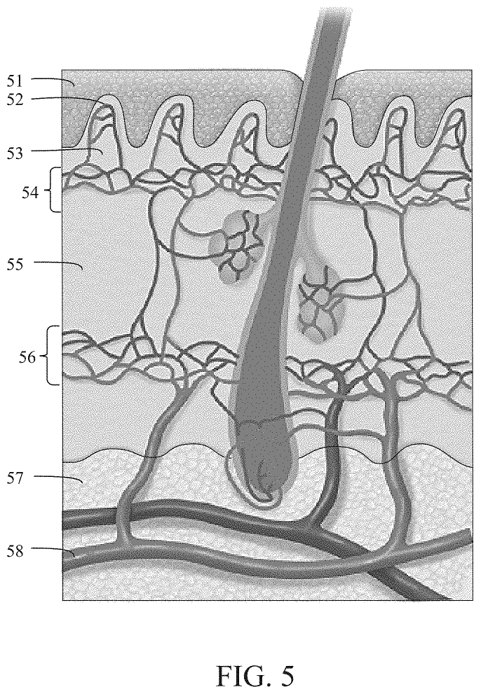

FIG. 5 is a pictorial representation of a cross-section of the skin illustrating the epidermis 51, the capillary loop system 52, the papillary dermis 53, the superficial vascular plexus 54, the reticular dermis 55, the deep vascular plexus 56, the subcutaneous fat 57 and the subcutaneous vasculature 58.

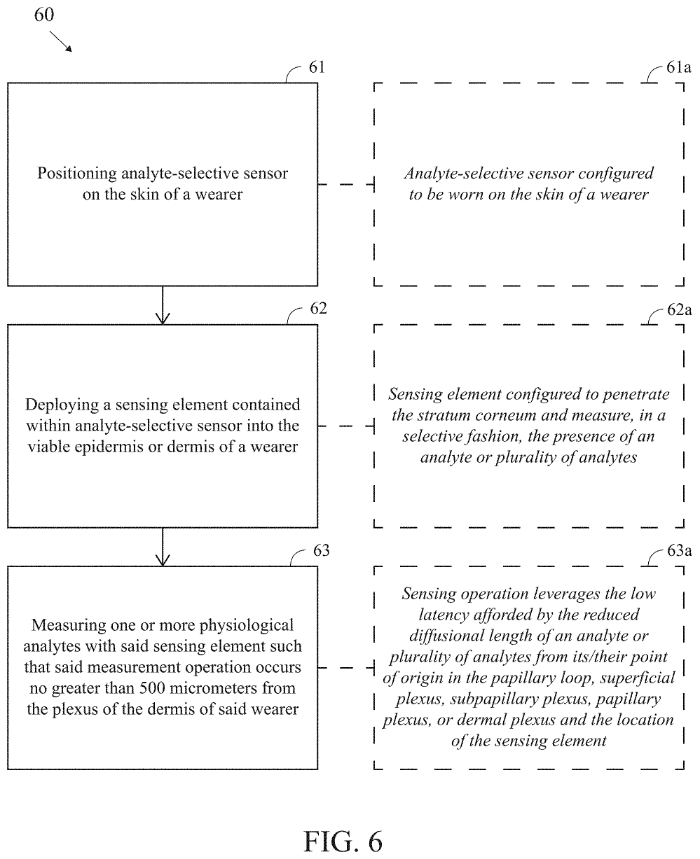

FIG. 6 is a process flow diagram illustrating a method 60 of the invention. At block 61, an analyte-selective sensor is positioned on the skin of a wearer. As stated in block 61a, the analyte-selective sensor is configured to be worn on the skin of the wearer. At block 62, the analyte-selective sensor is deployed such that a sensing element contained within the analyte-selective sensor penetrates the stratum corneum of the skin and becomes positioned in the viable epidermis or dermis of the wearer. At stated in block 62a, the sensing element is configured to penetrate the stratum corneum. At block 63, a voltage or current is applied at the sensing element and one or more physiological analytes is/are measured with the sensing element such that the measurement operation occurs no greater than 500 micrometers from the plexus of the dermis of the wearer. As stated in block 63, the sensing operation leverages the low latency afforded by the reduced diffusional length.

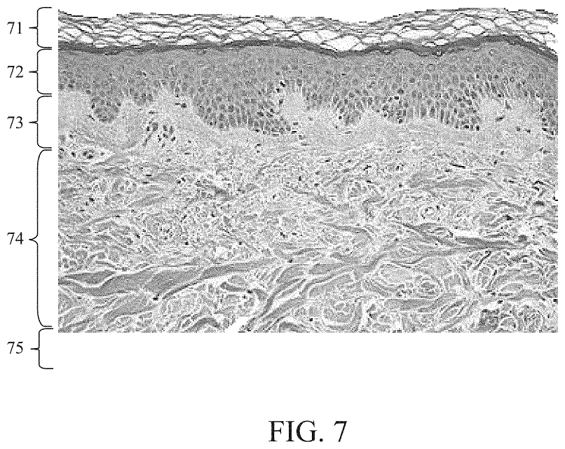

FIG. 7 is a pictorial representation of a cross-section of the skin delineating the anatomical location of the superficial and dermal plexus and structures contained therein including the stratum corneum 71, the epidermis 72, the papillary dermis 73, the reticular dermis 74 and the hypodermis 75.

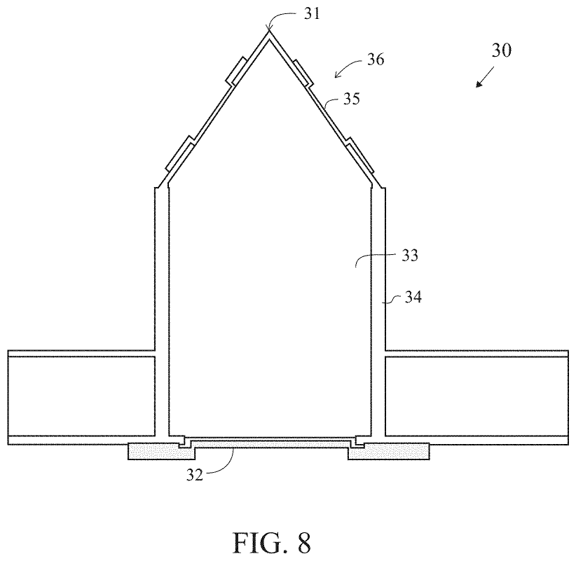

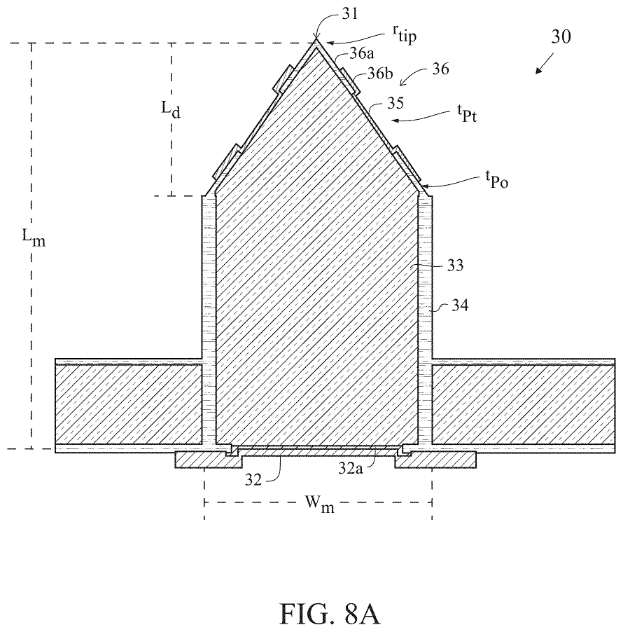

As shown in FIG. 8, each microneedle 30 of the microneedle array 20 preferably has a through-silicon via embedded within a microneedle 30. This allows the sensors to be individually probed as isolated constituents of the microneedle array 20. The microneedle array preferably can be reflow-soldered to nearly any circuit board in a manner similar to surface mount component technology (SMT). Each microneedle 30 preferably has an individual sensor 31 confined to a distal tip 36 of the microneedle 30, preferably in a region between 1 and 1500 μm from the distal end of the microneedle 30. The microneedle 30 preferably has a backside metal contact 32, a through needle VIA 33, insulation 34 to electrically isolate the microneedle 30 and a patterned metal contact 35 on the distal tip 36 of the microneedle 30.

As shown in FIG. 8A, the microneedle 30 preferably has insulation 34 composed of an oxide. The backside metal contact 32 is preferably composed of a nickel/gold material with an interior portion 32a composed of an aluminum material having a thickness ranging from 0.3 to 1 μm, and most preferably 0.5 μm. The microneedle 30 preferably has a through needle VIA 33 composed of a silicon material. The distal tip 36 preferably has oxide portions 36a and platinum portions 36b. The distal tip 36 preferably has an angle of approximately sixty degrees relative to a flat base of the microneedle 30. The thickness of the platinum portion 36b preferably ranges from 50 nanometers (nm) to 150 nm, and is most preferably 100 nm. The thickness of the oxide portion 36a preferably ranges from 0.5 to 1.5 μm, and most preferably 1 μm. The length, Lm, of the microneedle 30 preferably ranges from 200-2000 μm, and is most preferably 625 μm. The width, Wm, of the microneedle 30 preferably ranges from 100 to 500 μm, and is most preferably 160 μm. The distal tip 36 preferably has a length, Ld, ranging from 50 to 200 μm, and is most preferably 100 μm. The radius, Rtip, of the end of distal tip 36 is preferably 0.1 to 15 μm, and most preferably below 5 μm.

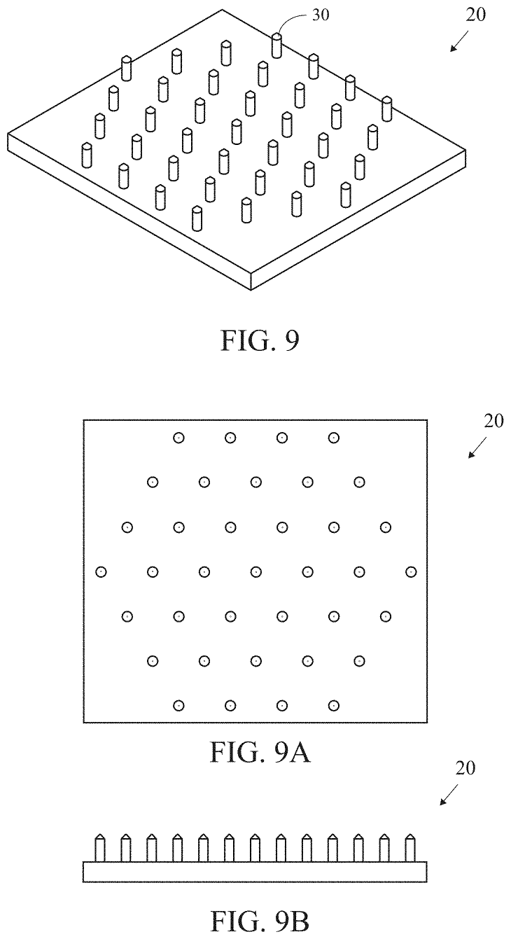

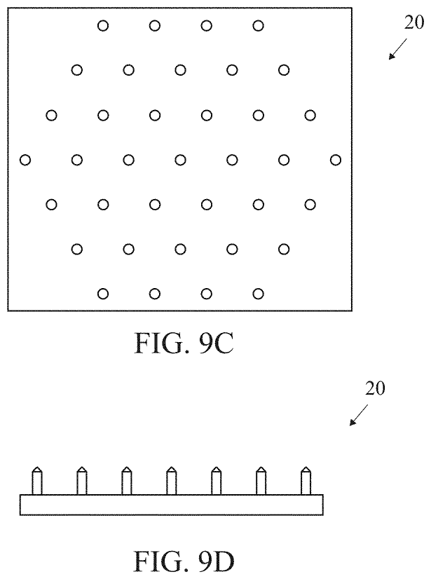

FIGS. 9-9D illustrate a microneedle array 20. The microneedle array preferably has from three to fifty microneedles 30, and more preferably from seven to forty microneedles 20.

McCanna et al., U.S. Pat. No. 9,933,387, for a Miniaturized Sub-Nanoampere Sensitivity Low-Noise Potentiostat System is hereby incorporated by reference in its entirety.

Windmiller, U.S. patent application Ser. No. 15/177,289, filed on Jun. 8, 2016, for a Methods And Apparatus For Interfacing A Microneedle-Based Electrochemical Biosensor With An External Wireless Readout Device is hereby incorporated by reference in its entirety.

Wang et al., U.S. Patent Publication Number 20140336487 for a Microneedle Arrays For Biosensing And Drug Delivery is hereby incorporated by reference in its entirety.

Windmiller, U.S. Pat. No. 10,092,207 for a Tissue Penetrating Electrochemical Sensor Featuring A Co Electrodeposited Thin Film Comprised Of A Polymer And Bio-Recognition Element is hereby incorporated by reference in its entirety.

Windmiller, et al., U.S. patent application Ser. No. 15/913,709, filed on Mar. 6, 2018, for Methods For Achieving An Isolated Electrical Interface Between An Anterior Surface Of A Microneedle Structure And A Posterior Surface Of A Support Structure is hereby incorporated by reference in its entirety.

PCT Application Number PCT/US17/55314 for an Electro Deposited Conducting Polymers For The Realization Of Solid-State Reference Electrodes For Use In Intracutaneous And Subcutaneous Analyte-selective Sensors is hereby incorporated by reference in its entirety.

Windmiller et al., U.S. patent application Ser. No. 15/961,793, filed on Apr. 24, 2018, for Heterogeneous Integration Of Silicon-Fabricated Solid Microneedle Sensors And CMOS Circuitry is hereby incorporated by reference in its entirety.

Windmiller et al., U.S. patent application Ser. No. 16/051,398, filed on Jul. 13, 2018, for Method And System For Confirmation Of Microneedle-Based Analyte-Selective Sensor Insertion Into Viable Tissue Via Electrical Interrogation is hereby incorporated by reference in its entirety.

Windmiller et al., U.S. patent application Ser. No. 16/701,784, filed on Dec. 3, 2019, for Devices And Methods For The Generation Of Alerts Due To Rising Levels Of Circulating Ketone Bodies In Physiological Fluids is hereby incorporated by reference in its entirety.

Windmiller et al., U.S. patent application Ser. No. 16/824,700, filed on Mar. 20, 2020, for Devices and Methods For The Incorporation Of A Microneedle Array Analyte-Selective Sensor Into An Infusion Set, Patch Pump, Or Automated Therapeutic Delivery System is hereby incorporated by reference in its entirety.

Windmiller et al., U.S. patent application Ser. No. 16/899,541, filed on Jun. 11, 2020, for a Mechanical Coupling Of An Analyte-Selective Sensor And An Infusion System And Information Conveyance Between The Same is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. A method for the measurement of at least one physiological analyte with reduced latency, said method comprising:
positioning an analyte-selective sensor on the skin of a wearer;
deploying said analyte-selective sensor such that a sensing element contained within said analyte-selective sensor penetrates the stratum corneum of the skin and becomes positioned in the viable epidermis or dermis of said wearer;
applying a voltage or current at said sensing element; and
measuring one or more said physiological analytes with said sensing element such that said measurement operation occurs no greater than 500 micrometers from the plexus of the dermis of said wearer.

2. The method claim 1, wherein said physiological analyte is an endogenous analyte or an exogenous analyte.

3. The method of claim 1, wherein said physiological analyte is a biomolecule, biomarker, metabolite, electrolyte, ion, hormone, neurotransmitter, protein, enzyme, co-enzyme, co-factor, vitamin, or mineral.

4. The method of claim 1, wherein said analyte-selective sensor is an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

5. The method of claim 1, wherein said analyte-selective sensor is a microneedle or a microneedle array.

6. The method of claim 5, wherein said microneedle or microneedle array possesses a vertical extent between 200 and 2000 μm.

7. The method of claim 1, wherein said sensing element is an electrode, transducer, detector, anode, or cathode.

8. The method of claim 5 wherein said sensing element is confined to a region between 1 and 1500 μm from the distal end of said microneedle.

9. The method of claim 1, wherein said plexus of the dermis is the papillary loop, superficial plexus, subpapillary plexus, papillary plexus, or dermal plexus.

10. A device for the measurement of at least one physiological analyte with reduced latency, said device comprising:
an analyte-selective sensor configured to be positioned on the skin of a wearer; and
a sensing element contained within said analyte-selective sensor configured to penetrate the stratum corneum of the skin and become positioned in the viable epidermis or dermis of said wearer such that said sensing element is located a spatial distance no greater than 500 micrometers from the plexus of the dermis.

11. The device claim 1, wherein said physiological analyte is an endogenous analyte or an exogenous analyte.

12. The device of claim 1, wherein said physiological analyte is a biomolecule, biomarker, metabolite, electrolyte, ion, hormone, neurotransmitter, protein, enzyme, co-enzyme, co-factor, vitamin, or mineral.

13. The device of claim 1, wherein said analyte-selective sensor comprises an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

14. The device of claim 1, wherein said analyte-selective sensor comprises a microneedle or a microneedle array.

15. The device of claim 14, wherein said microneedle or microneedle array possesses a vertical extent between 200 and 2000 μm.

16. The device of claim 1, wherein said sensing element is an electrode, transducer, detector, anode, or cathode.

17. The device of claim 14 wherein said sensing element is confined to a region between 1 and 1500 μm from the distal end of said microneedle.

18. The device of claim 1, wherein said plexus of the dermis is the papillary loop, subpapillary plexus, papillary plexus, or dermal plexus.

19. The device of claim 16 wherein said electrode is confined to a region between 1 and 1500 μm from the distal end of said microneedle
